(19) United States
(12) Patent Application Publication
(10) Pub. No.: US 2016/0095518 A1
(43) Pub. Date: Apr. 7, 2016

(54) System for Remote Diagnosis of a Stroke

(71) Applicant: VRIJE UNIVERSITEIT BRUSSEL, Brussel (BE)

(72) Inventors: Andre CONVENTS, Diegem (BE); Raf BROUNS, Londerzeel (BE); Rohny VAN DE CASSEYE, Hoeilaart (BE); Alexis VALENZUELA ESPINOZA, Vorst (BE); Robbert-Jan VAN HOOFF, Elsene (BE)

(73) Assignee: Vrije Universiteit Brussell, Brussel (BE)

(21) Appl. No.: 14/786,671

(22) PCT Filed: Apr. 23, 2014
(86) PCT No.: PCT/EP2014/058239

§ 371 Date: Oct. 23, 2015

(30) Foreign Application Priority Data
Apr. 23, 2013 (BE) 2013/0287

(51) Int. Cl. A61B 5/00; A61B 7/04; A61B 3/113; A61B 5/0404; A61B 5/0205

(52) U.S. Cl. CPC A61B 5/0022; A61B 5/0404; A61B 5/0205; A61B 5/0077; A61B 3/113; A61B 5/0013; A61B 7/04; A61B 2560/0431; A61B 2560/0214; A61B 2560/0475

(57) ABSTRACT

A portable system for remote diagnosis of a stroke is arranged in a housing provided with a handgrip and comprising audio recording and reproduction means for recording and reproducing sound at the location of a patient. The portable video recording means for recording at least eye and foot movements of the patient is arranged to be set up in such a way that the patient can be captured from head to toe. Measuring means to determine at least a blood glucose value in the patient, and communication means for forwarding the measured blood glucose value and the recorded movements and sound via a wireless network from the location of the patient and for receiving audio data originating from a location where a doctor is provided.

### FIELD OF THE INVENTION

The present invention is generally related to the field of systems for remote diagnosis, and more particularly for remote diagnosis of strokes.

### BACKGROUND OF THE INVENTION

Strokes represent a significant health problem worldwide. In Belgium, 20,000 people suffer a stroke every year. The only treatment proven to increase the chance of good recovery is treatment with a clot-busting drug (thrombolysis). Since it has been demonstrated that time is crucial in this treatment, any procedure allowing the treatment to begin earlier will increase the likelihood of a good recovery. Studies have shown that telemedicine is a good, accurate and reliable technique for reducing the time between the occurrence of the symptoms of the stroke and the treatment thereof. The optimum treatment of a stroke must be implemented in a continuous care path that begins pre-hospital, is continued in hospital and ends with rehabilitation. If the pre-hospital focus is on stabilisation of vital parameters such as blood pressure, sugar level, oxygen level or temperature, the stroke can be treated much more effectively in hospital.

Consequently, there is a clear need for an accurate and reliable treatment that can reduce the time between the occurrence of the symptoms of the stroke and the beginning of the treatment.

A number of research projects have been described in the medical literature relating to the treatment in situ of a stroke. A number of hospitals (e.g. in remote rural areas) are often connected therein to one hub. As in this invention, the ultimate aim is to increase the number of patients who are thrombolysed and to investigate whether telemedicine can reduce the treatment time.

Systems for telemedicine in general are well known in the prior art. A large amount of patent literature also exists in this technical domain. An older patent application such as JP11033000 describes a medical terminal for an ambulance. The proposed system includes a part for entering data and a measuring part which comprises a number of measuring instruments.

Some systems for interconnecting an emergency vehicle and a medical establishment are satellite-based, such as, e.g. J2004/141558.

Patent U.S. Pat. No. 6,925,357 relates to a robotic system for medical applications. The system comprises, inter alia, a camera and a monitor to allow the care giver to care for a patient in situ through the robot.

Application US2006/122466 relates to a modular telemedicine system with a universal adapter that connects diagnostic, identification and audiovisual communication function modules to a variable process module that performs data transmission, data processing and output.

In US2008/312519, an examination unit is disclosed which has an integrated mini-laboratory analysis unit. The system comprises an ultrasonic device, a patient monitor, an ECG, a device for monitoring the vital functions and/or for immediate medical treatment of patients with symptoms of acute cardiovascular disease, myocardial infarction, a stroke, etc. Biochemical or cell-biology investigation of blood samples can be carried out with the mini-laboratory analysis unit.

In the paper ‘Feasibility of Prehospital Teleconsultation in Acute Stroke—A Pilot Study in Clinical Routine’ (Bergrath et al., PLoS ONE, Vol.7, No.5, e336796, May 2012) a solution is disclosed wherein an ambulance is equipped with a portable data transmitter unit. This allows broadband communication via four parallel data channels from different network providers. A vital data monitor, a digital camera to take still pictures and headsets for audio communication and a video camera are provided connected to the transmitter unit. Two-way audio communication is possible. The video camera is used for evaluating the neurological status. The video camera is embedded into the ceiling of the ambulance and allows an optical zoom to the patient's face and looking at all body regions from the teleconsultation centre. Video streaming is conducted both in the phase the ambulance is still on-scene and on the way to the hospital. The video data is not recorded. A major drawback of this solution is that the system can only be used inside the ambulance.

The paper ‘TELEBAT: Mobile Telemedecine for the Brain Attack Team’ (LaMonte et al., Journal of Stroke and Cerebrovascular Diseases, Demos Publications, New York, Vol.9, No.3, May 2000, pp.128-135) is also concerned with shortening the time to treatment once a patient is known to the emergency medical system. It exploits wireless transmission technology to send image and clinical data from the ambulance to the hospital. The system is intended for ambulance installation and comprises digital camera, television, microphone, video cassette recorder, equipment to monitor vital signs, a hand-held blood laboratory device and a video and communication system that integrates camera inputs while managing a parallel array of 4 digital cellular phones.

U.S. Pat. No. 5,544,649 provides an example of patient health monitoring system that is not portable. The patient is monitored by a health care worker at a central station, while the patient is at a remote location. Cameras are provided at the patient's remote location and at the central station such that the patient and the health care worker are in interactive visual and audio communication. A communications network such as an interactive cable television is used for this purpose. Various medical condition sensing and monitoring equipment are placed in the patient's home, depending on the particular medical needs of the patient. The patient's medical condition is measured or sensed in the home and the resulting data is transmitted to the central station for analysis and display. The health care worker then is placed into interactive visual communication with the patient.

The aforementioned documents from the prior art all have one or more significant disadvantages. Some are too complex or too cumbersome to use. Many prior art systems can only be built in into an ambulance and do not lend themselves to use outside the ambulance.

As previously indicated, it is of the greatest importance that a diagnosis can be carried out as quickly as possible. However, none of the aforementioned prior art systems is specifically adapted to take this objective substantially into consideration.

There is then also a need for a telediagnosis system, in particular telediagnosis of strokes, which is designed in such a way that the fast interaction between the patient/ambulance on the one hand and the doctor (neurologist)/hospital on the other hand is possible. By means of a system of this type, the relevant specialist should be able to perform a diagnosis remotely and there is therefore no longer a need for a specialist in situ. There is also a need for a system for diagnosis which is relatively inexpensive and can be installed in an ambulance and possibly taken along to a hospital bed.

### SUMMARY OF THE INVENTION

It is an object of embodiments of the present invention to provide for a portable mobile system for remote diagnosis that is user-friendly and allows the necessary data to be collected quickly in order to be able to perform a diagnosis.

The above objective is accomplished by the solution according to the present invention.

In a first aspect the invention relates to a portable system for remote diagnosis of a stroke, arranged in a housing and provided with a handgrip and comprising

audio recording means for recording sound of a patient and audio reproduction means for reproducing sound for said patient,

portable video recording means for recording at least eye movements and foot movements of the patient, said portable video recording means arranged to be set up in such a way that the patient can be captured from head to toe,

measuring means to determine at least a blood glucose value in said patient,

communication means for forwarding said measured blood glucose value and said recorded movements and sound of the patient via a wireless network from the location of the patient and for receiving at least an audio signal originating from a location where a doctor is present.

The proposed portable system permits a wireless connection to be set up between the doctor, e.g. a neurologist in the case of a stroke, and a patient. The portable video recording means can be set up in such a way that the patient can be captured from head to toe. The doctor can thus perform a stroke scale remotely, wherein, inter alia, foot and leg movements and movement of the eyes and the eyelids can be observed. The system is further equipped for determining a blood glucose value and for forwarding this measurement value via the communication means. It is crucial for the neurologist to have this data available as soon as possible. A major asset of the proposed system is its portability. The system is arranged in a housing provided with a handgrip. This allows employing the system not only in the ambulance, but also on a location outside the ambulance. The handgrip can in certain embodiments be embedded in said housing or in other embodiments be part of the communication means or another system component.

In a most preferred embodiment the communication means are also configured to receive a video signal originating from the location where the doctor is present. The portable system comprises a monitor for visualising the images originating from the location of the doctor. This has the great advantage that the patient can then also see the doctor.

In a preferred embodiment the angle of view is at least 80° and, in a most preferred embodiment, the angle of view is at least 95°. In its simplest form this can be achieved by means of a camera that can cover this wide angle. An alternative is the use of a camera that covers a narrower angle but rotates mechanically through 80°, manually under remote control or automatically. A further alternative is the use of two to three different cameras pointed at different parts of the body and each covering a smaller part of the angle of view.

In a further embodiment the portable system comprises a monitor which can be set up in such a way that the surface of the screen forms at least a 10° angle with the surface in which the camera is positioned. As a result, the appliance can be suspended on the roof of the ambulance and on a hospital bed so that the patient sees the monitor and at the same time the camera covers the eyes and the feet. An embodiment is preferred wherein the angle between the image surface and the camera is at least 20°. In this embodiment, the patient will see the doctor without having to move his neck/head; as a result, the doctor is able to analyse the patient in a natural posture.

In a different embodiment, the portable system further comprises measuring means to determine at least the blood pressure and/or oxygen saturation in the blood (SPO₂) and the communication means are equipped to forward the obtained measurement values. The proposed system thus permits a set of vital parameters to be forwarded, preferably more or less simultaneously, and enables a doctor or other qualified person to monitor a number of data relating to the patient remotely in real time. The vital parameters are, inter alia, the blood glucose level, blood pressure, oxygen saturation in the blood (SPO₂), cardiac rhythm, age and gender of the patient. Useful additional information is provided by the electrocardiogram and the clotting level of the blood.

In a preferred embodiment the communication means are equipped to forward the video images of the patient with an image resolution of at least 160×120 pixels and a picture rate of at least 4 frames/second.

By selecting parameters in these specific ranges for the video connection, it becomes possible to obtain an audiovisual signal of a quality such that it becomes possible for the doctor in fact to perform a good diagnosis remotely. In a more specific embodiment said communication means are further equipped to carry out an image compression of at least 50%. This permits the important image information to be forwarded wirelessly with the current prior art.

In order to enhance the system autonomy and portability the system comprises in a preferred embodiment at least two independent batteries. In one embodiment at least one battery is provided with a voltage decharge controller to keep the voltage from dropping under a given threshold value.

In another preferred embodiment the system is arranged for simultaneously activating at least said audio recording means, said portable video recording means and said communication means.

In one embodiment the system is further provided with a reader for an electronic identity card. In this way, patient data can quickly and simply be entered and forwarded to the hospital.

In one embodiment the measuring means are further equipped to record an electrocardiogram and/or determine the clotting level of the blood, wherein the communication means are equipped to forward the obtained results.

In one embodiment the system is provided with a processing unit configured to provide information on the health of said patient on the basis of measurements of at least two parameters. The processing unit is preferably equipped to perform a stroke scale. The responses of the patient can then be entered immediately and forwarded to the doctor.

In a preferred embodiment the portable system according to this invention weighs less than 7 kg.

In a preferred embodiment the portable system comprises a tripod for attaching the video recording means.

In a preferred embodiment the portable system comprises a stable snap-on system which can be attached to an arm (preferably at least 20 cm in length). In a more specific embodiment this snap-on system is a standard vesa plate.

In a preferred embodiment the system comprises an arm (preferably at least 20 cm in length) which has a snap-on system to be snapped into place on a 32 mm round rod. In this way, the portable appliance can be snapped into place on the roof of an ambulance, and also on the side of a hospital bed.

The invention further relates also to a kit which comprises a portable system as described above. Further components of the kit may be one or more measuring means to determine vital parameters, or a portable computer.

In another aspect the invention relates to a portable system for remote diagnosis of a stroke, comprising

audio recording means for recording sound of a patient and audio reproduction means for reproducing sound for said patient,

portable video recording means for recording at least eye movements and foot movements of said patient, said portable video recording means arranged to be set up in such a way that the patient can be captured from head to toe,

communication means for forwarding said recorded movements and sound of said patient via a wireless network from the location of said patient and for receiving at least an audio signal originating from a location where a doctor is present, said communication means also being configured to receive a video signal originating from the location where the doctor is present,

a monitor for visualising said image originating from the location where the doctor is present, whereby said monitor can be set up in such a way that the surface of said monitor forms an angle of at least 10° with the surface of said video recording means.

In a preferred embodiment the angle of view is at least 80° and, in a most preferred embodiment, the angle of view is at least 95°. In its simplest form this can be achieved by means of a camera that can cover this wide angle. An alternative is the use of a camera that covers a narrower angle but rotates mechanically through 80°, manually under remote control or automatically. A further alternative is the use of two to three different cameras pointed at different parts of the body and each covering a smaller part of the angle of view.

In a preferred embodiment the portable system comprises a handgrip with a handle. The handgrip can be included as a part of the monitor.

For purposes of summarizing the invention and the advantages achieved over the prior art, certain objects and advantages of the invention have been described herein above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

The above and other aspects of the invention will be apparent from and elucidated with reference to the embodiment(s) described hereinafter.

### BRIEF DESCRIPTION OF THE DRAWINGS

The invention will now be described further, by way of example, with reference to the accompanying drawings, wherein like reference numerals refer to like elements in the various figures.

FIG. 1 illustrates the angle of view of the video recording means of the portable system according to the invention.

FIG. 2 shows an embodiment of the proposed portable system for remote diagnosis of a stroke.

FIG. 3 shows a monitor on which vital information is displayed which is forwarded via the wireless connection.

FIG. 4 illustrates a front view of an embodiment of the portable system.

FIG. 5 illustrates a side view of the embodiment from FIG. 4.

FIG. 6 illustrates an embodiment of the grip arm.

### DETAILED DESCRIPTION OF ILLUSTRATIVE EMBODIMENTS

The present invention will be described with respect to particular embodiments and with reference to certain drawings but the invention is not limited thereto but only by the claims.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

It should be noted that the use of particular terminology when describing certain features or aspects of the invention should not be taken to imply that the terminology is being re-defined herein to be restricted to include any specific characteristics of the features or aspects of the invention with which that terminology is associated.

In the description provided herein, numerous specific details are set forth. However, it is understood that embodiments of the invention may be practiced without these specific details. In other instances, well-known methods, structures and techniques have not been shown in detail in order not to obscure an understanding of this description.

The present invention relates to a system for use in telemedicine. The term ‘telemedicine’ means the remote application of care provision and support using information and communication technology, geared towards the primary care process, so that the quality of life of the care user increases. Known forms of telemedicine are telemonitoring in the case of traumas, wherein information relating to vital parameters is forwarded to the hospital, so that the latter is better prepared for the arrival of the patient, and specific forms of telediagnosis, wherein the patient is in contact with his doctor via a digital platform and is thus assisted. The present invention is to be placed in the domain of telediagnosis.

It may be expected that the need for such solutions will only increase in future. There are in any event various significant associated advantages, inter alia with the aim of achieving constantly improving quality of care and a more effective use of care professionals.

Wireless communication systems are increasingly used in technological resources for telemedicine in general. For treatment of this type, it is important that the coverage provided via wireless communication is almost complete. The current 3G technology already achieves a coverage of 97%. The rollout of 4G networks is well underway. However, availability is known to be variable. For this reason, it is necessary to have a system that is as robust as possible and can operate in all conditions.

The present invention is based on the insight that substantial timesaving can be achieved by allowing as many tasks/actions which do not necessarily have to be performed in the hospital to take place already in a pre-hospital phase. The pursuit of this aim defines the technical requirements of the system according to the invention.

The portable system according to this invention aims to achieve direct communication via a wireless connection between the ambulance or another location where the patient is present on the one hand and, on the other hand, the doctor (neurologist) at another location (e.g. in the hospital or at home) with the aim of supporting the final diagnosis. The established communication link permits at least two-way transmission of audio data, and also the transmission of video data from the location where the patient is present to the other location. The doctor can thus observe the patient. In a most preferred embodiment of the invention the video traffic is also two-way, so that the patient can also see the doctor on a monitor which forms part of the telediagnosis system.

Stabilisation and possible correction of anomalous vital parameters in consultation with the patient's neurologist is also necessary in order to guarantee a (more) positive outcome for the patient. In a preferred embodiment, these vital parameters are: heart rate, blood glucose level, blood pressure, oxygen saturation in the blood (SPO₂), age and gender of the patient. In a specific embodiment, blood coagulation and an electrocardiogram (ECG) are added. In a preferred embodiment, the portable telemedicine system is equipped to provide the neurologist simultaneously and in real time with vital parameters.

The telemedicine system according to the invention is furthermore mobile and compact, as a result of which it has a ‘plug & play’ character. It can easily be used in any ambulance (e.g. by means of a snap-on system with a rigid hydraulic grip arm) and can be started with a press of the button. Important requirements are that the system is user-friendly and does not ‘hang’ in the way of the (para)medical personnel in the ambulance.

The proposed system forwards image and sound of the patient, so that the doctor (neurologist) is able to perform a stroke scale remotely in a pre-hospital phase. The NIHSS (National Institutes of Health Stroke Scale) stroke scale has hitherto been the most generally used scale for assessing the severity of the stroke. However, a number of significant disadvantages are associated with this scale. An expert medically trained person must be physically present to perform the test. The examination requires 8 to 10 minutes on average. Furthermore, it is a complex scale and some parts are difficult to reproduce. A number of relevant data, such as speech and movement of the hands and feet, are then taken relatively little, or not at all, into account. An improved stroke scale is proposed here, which is referred to below as the UTSS scale, wherein UTSS stands for ‘Unassisted TeleStroke Scale’. The UTSS stroke scale is optimised to carry out a remote diagnosis of a stroke and the severity thereof via the video connection. The main improvement of the new stroke scale is that no intervention is required from personnel at the patient's bedside. A binary response (e.g. yes or no) goes with each element in the test. All the actions which the patient must carry out can also be carried out in the restricted environment of an ambulance while the patient is strapped to the ambulance bed/stretcher. The patient has very limited possibilities for moving his arms and cannot move his legs. The UTSS scale can be performed in only around three minutes. In other words, an essential timesaving of some 5 minutes is achieved.

In order to be able to perform this stroke scale, it is nevertheless of prime importance that the quality of the audio and video permits the diagnosis to be performed effectively. In performing the stroke scale, the patient is asked, for example, to move his eyes from left to right or to close his eyes tightly. Involuntary eye movements are also important observations for the treating neurologist. The patient is also asked to perform specific vertical arm movements which are possible in an ambulance. Given that a patient's legs are immobilised in an ambulance, attention must be focused on the feet in order to test the motor activity of the lower limbs. In order to carry out a diagnosis, it must of course be possible for the doctor to observe all of this remotely.

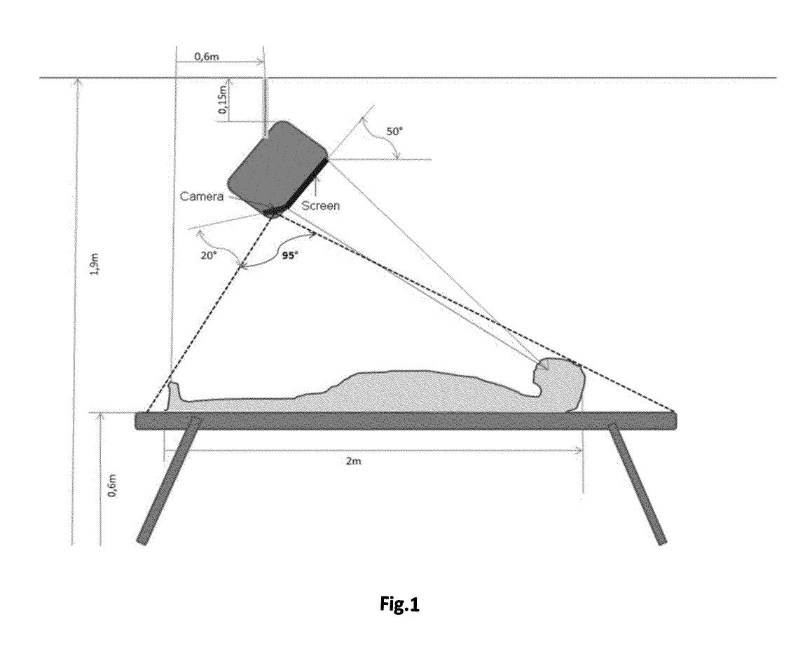

To make this possible, the portable system for remote diagnosis according to the present invention provides a camera to record images of the patient in order to then forward them via the wireless connection to the doctor. The camera can be set up in such a way that the patient can be viewed from head to toe by the doctor. In one advantageous embodiment the camera has an angle of view of at least 80°, preferably at least 90° and most preferably even at least 95°. As a result, the patient is in full view of the doctor at the other end of the connection. FIG. 1 provides an illustration wherein, along with the angle of view, a number of dimensions and the angle of the monitor are also indicated by way of example.

The camera must provide an angle of view of at least 90° and preferably offer a resolution of at least 3.0 Megapixels. In a preferred embodiment, the camera will have an angle of view of more than 95°. If the camera is then suspended at a height of at least 75 cm above the patient and 60 cm from the patient's feet in the direction of the head (horizontal surface), a person measuring 2 metres from head to toe can be captured. Due to the resolution of at least 3.0 Megapixels, the doctor is able to see the eyes of the patient to a sufficient extent in order to be able to establish any anomaly. In an advantageous embodiment the camera is a so-called fisheye camera, i.e. a camera with a lens system with a short focal length that produces a strong wide-angle effect. Some examples of cameras that can be used are the Mobotix Q24, Mobotix S14, Axiss M3006 and Axiss M3007. In an advantageous embodiment an IP camera is applied.

The audio connection is two-way. Both parties must at least be able to hear one another clearly. There must be no significant discrepancy between the image that the doctor sees and the sound (in order to be able to analyse lip movements during speech). An audio system that filters ambient noise and can focus on the patient's voice is to be preferred. The patient must be able to hear the doctor, even if the ambulance sirens are activated. A good amplification of the doctor's sound is desirable for this purpose. Since the portable appliance is integrated with the UTSS scale which relies strongly on verbal interaction between the neurologist and the patient, the audio system must be able to make both parties sufficiently understandable to one another.

Furthermore, the question arises concerning the video settings which, on the one hand, still offer a good quality so that the performance of a diagnosis without medical expertise at the patient's bedside remains possible, but, on the other hand, also limit transmission overhead as much as possible and are cost-effective. Extensive tests have been carried out in order to determine suitable combinations of parameter settings. A video connection via wireless communication depends on the illumination in the room (typically expressed in Lux), the resolution of the image (expressed in pixels), the number of images captured per second (frames or pictures per second), the compression of the full video signal in order to enable the transmission via wireless location (as a percentage). An optimum combination of these elements is of fundamental importance for performing the diagnosis with the portable appliance.

Well known formats or types of picture which possibly come into consideration are, inter alia, the VGA (i.e. a picture with 640×480 pixels), ‘Common Intermediate Format’ (CIF) and a PDA (Personal Digital Assistant) format. Inter alia, a CIF format with a resolution which is ¼ of a VGA picture (i.e. 320×240 pixels) and a PDA with a further quarter lower resolution (160×120) have been investigated. The neurologist has the option of choosing the picture quality himself

A picture compression technique is applied to the pictures. Various such techniques are known to the person skilled in the art. The MPEG compression algorithm is, for example, well known in the prior art. For the average person skilled in the art, it is clear that other picture compression techniques from the prior art are also immediately applicable instead of MPEG in the context described here. It is assumed that maximum compression will always be sought without loss of diagnostic information.

A further factor which is of importance relates to the luminous intensity at the location where pictures are taken. It may be clear that luminous intensity plays an important part, e.g. if the test already described is carried out with the movement of the eyes. A wide range from very dark to very light is passed through in order to find a suitable value or subrange of values which deliver an acceptable result. From 20 lux, the picture is sufficiently clear for diagnostic purposes.

Furthermore, a choice must also be made regarding the frame rate (i.e. the picture rate). Obviously, a higher picture rate also means a higher required bit rate in order to forward all this information. This applies above all to the measurements of eye movements.

The performance of numerous tests has resulted in the surprising realisation that a low resolution can suffice for a neurological analysis. The following combinations of parameters represent the minimum requirement, desired values and highly advantageous values respectively.

As a lower limit, it can be assumed that the picture must have a minimum resolution of 160×120 pixels. The applied amount of MPEG compression is 70% and the frame rate is at least 4 frames per second. As far as luminous intensity is concerned, values of as little as 20 to 50 lux appear sufficient in order to maintain sufficient interaction in the telemedicine system. The created bit stream is then approximately 70 kbps. In order to obtain a good video quality, it is most appropriate to have a resolution of 320×240 pixels and a frame rate of 6 frames/second, still with an MPEG compression of 70%. However, a bit rate of around 400 kbps is then necessary. Obviously, parameter settings requiring even higher bit rates deliver very good quality. A favourable combination of this type may, for example, be a picture resolution of 640×480 pixels, with 70% MPEG compression and a frame rate of 6 frames/s. The connection must then be able to provide a bit rate of 1.6 Mbps. If the aforementioned bit rates are compared with what is technically feasible, e.g. in a 3G network (maximum upload speed of 2 Mbps and maximum download speed of 7.2 Mbps), it becomes clear that the proposed parameter combinations are in every case technically feasible over a 3G network.

Tests reveal that the raw fish-eye image from a 360° camera provides an adequate image for being able to detect movement of the eyes from the point where a resolution of 3 pixels/cm² on the subject is achieved.

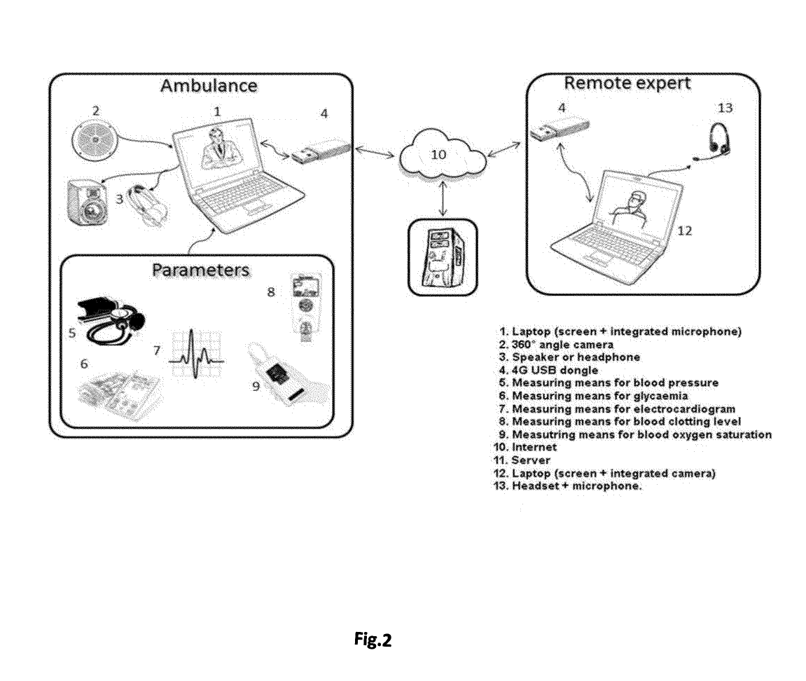

FIG. 2 shows an embodiment of the system according to the present invention. Note that the housing is not shown in the figure.

As indicated, in order to achieve a substantial timesaving in the interval between the stroke and the treatment, it is similarly extremely desirable to collect a number of vital parameters already in the pre-hospital phase and to forward them to the hospital's data management system. In a preferred embodiment, the system according to the invention is therefore further provided with a number of hardware components for determining these vital parameters.

Specifically, the proposed solution comprises at least measuring means for determining blood glucose levels in the patient. The glucose must be measured and forwarded as quickly as possible to the neurologist. To do this, a system is required that can forward its value quickly and at the same time causes little additional inconvenience for the nurse. The glucose can, for example, be measured with a normal glucose meter as part of the standard protocol of the emergency nurses. Furthermore, there are also software applications for carrying out the measurements using a smartphone, appliances for wireless measurement, etc. Some glucose meters have the facility for communication via Bluetooth. In a specific design, the blood glucose meter is integrated into the portable appliance.

Furthermore, the portable system according to the invention further preferably comprises measuring means for determining one or more of the following parameters: the heart rate, blood pressure, INR (International Normalized Ratio, i.e. the blood clotting level), SPO₂ (the blood oxygen saturation), glycaemia, electrocardiogram (ECG). These values can be determined with standard means known from the prior art. For the sake of completeness, these are summarised briefly below.

Various commercial solutions are available for performing an ECG. Solutions are known wherein the measurement data can be forwarded wirelessly. In WO2004/02301 a wireless ECG system is described which connects all electrodes to a box on the patient's arm. This box is in wireless contact with a central monitor via a Bluetooth connection.

A finger capsule is typically used to measure the blood oxygen saturation (SPO₂). These appliances are usually referred to as a pulse oximeter. Examples of these include: Concord Pulse Oximeter or Pulse Oximeter TD-8201.

For a non-invasive measurement of blood pressure, many options are generally known, often based on the use of sensors. Wireless solutions are also well known.

The heart rate can be measured with the conventional methods. Some more innovative solutions use e.g. a smartphone.

Mobile appliances are commercially available for measuring an INR value.

The system furthermore comprises a tool for direct communication with the hospital. From 2-lead to 12-lead ECGs are forwarded via this route.

In one embodiment the system automatically forwards the following blood values to the neurologist: INR value, glycaemia and also a general complete blood count. Furthermore, the neurologist at the other end of the connection is preferably able to monitor the following vital parameters quickly (e.g. in real time): blood pressure, blood saturation, heart rate and ECG. All the instruments are preferably integrated into the portable appliance, either as individual instruments in a housing or as a fully integrated appliance with an overall housing.

As indicated the system comprises of various components, like PC, camera and medical device(s). In order to achieve its autonomy and portability the proposed system has major energy requirements for a long time-period. This cannot be supplied by for instance a mobile phone. With the multitude of system components at least two batteries are needed as well as integration for user-friendly charging of the system. Based on learnings with ambulance personnel high energy needs are required, as an ambulance is an environment with unstable energy supply. This implies a combination of two or more batteries. As an example a combination is needed of battery components of a DELL PC Li-ion battery of 42Whr-11.1VDC and a Li-ion -Yanec battery of 6,7Ah 24VDC to support both PC and IP camera. In addition, other battery components are preferably added, such as a Li-ion—Yanec battery of 6,7Ah-16VDC for a router.

To extend the battery life, a voltage decharge controller is used, so that the battery voltage cannot drop under a selected voltage between 18VDC and 21VDC. In addition the system synchronises the simultaneous start-up of the PC, video and audio.

To enable stability of battery reloading of multi-battery components a voltage dip device has been developed which can be fixed to prevent battery drains between 18VDC till 21VDC. In addition, the integrated system enables simplified and synchronized activation by integrating a connection component (relay) to enable activation of video, audio, computer components and medical devices at the same time.

The portable system according to the present invention furthermore preferably comprises a reader for an electronic identity card. In an advantageous embodiment, this can form a separate unit, but if required may also comprise an integrated component of the system. Patient data can thus be forwarded immediately to the hospital.

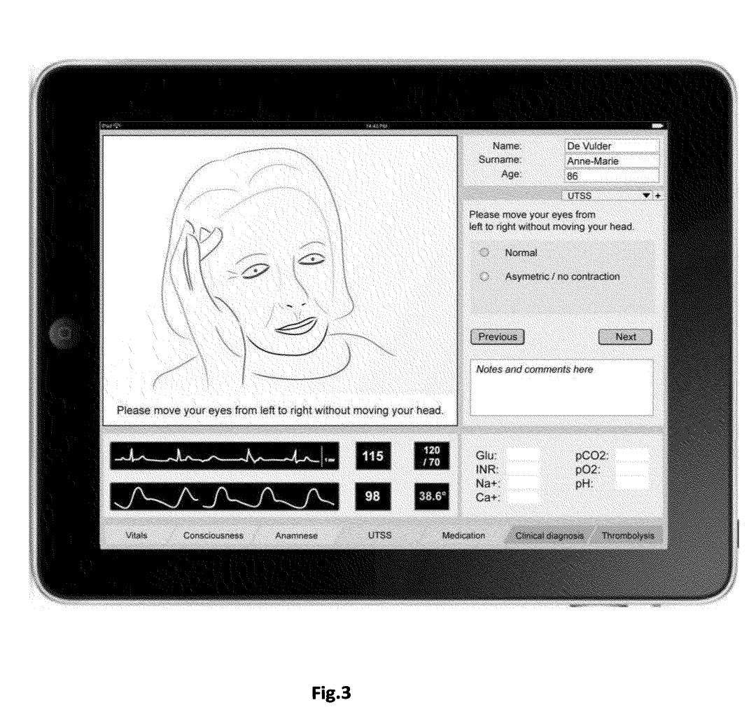

As previously mentioned, the system comprises a monitor which can be installed in the ambulance. In an advantageous embodiment this monitor collects, analyses and displays the vital information. The mobile system in the ambulance is equipped to forward data wirelessly and continuously to a clinical workstation in the hospital. It is possible to view the exchanged data during transport or thereafter. FIG. 3 provides an illustration.

As already indicated, the camera forms an essential component of the system, in particular for performing a stroke scale. In an advantageous embodiment the camera can be inserted in a holder fixed to the ceiling of the ambulance, so that a stable image is ensured. Such simple and cost-effective solutions have already proven their validity. The screen can preferably be folded away, most preferably in such a way that the camera is then covered. In specific embodiments, the screen can be attached to the ceiling of the ambulance, while in other embodiments it is suspended on a tube, so that it can be positioned closer to the patient. The screen may be such that no interaction is possible, or may combine a plurality of functionalities. In this last case, an implementation with a tablet PC is possible. The tablet can then be removable. The nurse can then easily enter data, consult patient history and, if necessary, make contact with a specialist.

It is important that the patient understands the doctor completely in situ. It is therefore extremely necessary also to provide audio as already explained above. In one embodiment a loudspeaker and a microphone are also integrated into the camera arm.

In one embodiment the system comprises a microprojector to project the neurologist onto the ceiling of the ambulance. As a result, it is only necessary to provide a space on the ceiling onto which the projection can take place.

The activation of the telestroke system can be performed in different ways. A first option is by means of a push-button on the screen of a smartphone. Alternatively, the push-button may be positioned e.g. on an armband.

In one embodiment of the system the server, monitor and camera are integrated. In an alternative, the server and camera are integrated and the monitor is separate. As already mentioned, the monitor can also be replaced with a tablet PC, possibly with a built-in camera. Further options are that all components of the system are modular or that the server and screen are integrated and that the camera remains separate.

As far as the arrangement of the instruments for measuring the clinical data such as vital parameters are concerned, it is chosen in some embodiments to accommodate all appliances in the same housing. In a different embodiment, everything is kept separate. Alternatively, for example, the appliance for determining the general blood values and the appliance for measuring the INR value can also be accommodated together in a single housing.

An important aspect necessary for achieving a good performance with the proposed solution relates to the software for guiding the neurologist through the telediagnosis, i.e. the graphical user interface. Everything must be geared towards enabling fast working The software also comprises the necessary intelligence to be able to propose the appropriate decisions on the basis of real-time monitoring, blood analysis results and input obtained in the performance of the stroke scale. One of these decisions may, for example, be that the patient is eligible for a thrombolysis or a different treatment for an acute stroke. During the decision support, the software can alert the neurologist e.g. if specific vital parameters attain alarming values. The software can also support the dialogue between the patient and the doctor. The connection to the hospital via the audio/video connection and via the data connection is obviously of vital importance for this purpose. The software allows all the necessary steps to be run through in order to reach a diagnosis. This reduces the risk of the doctor skipping a crucial step or missing crucial information. The software supports the doctor in the entire process, as a result of which the error margin will be reduced and the doctor can reach a decision more quickly. The software will also enable the doctor to perform the stroke scale remotely, and automatically calculates the total score of the scale on the basis of the doctor's input. On the basis of the information obtained, the doctor can take a well-considered and informed decision concerning the allocation or otherwise of a specific therapy such as e.g. thrombolysis. The supporting software will also use the collected information to make ‘outcome predictions’ for a specific patient.

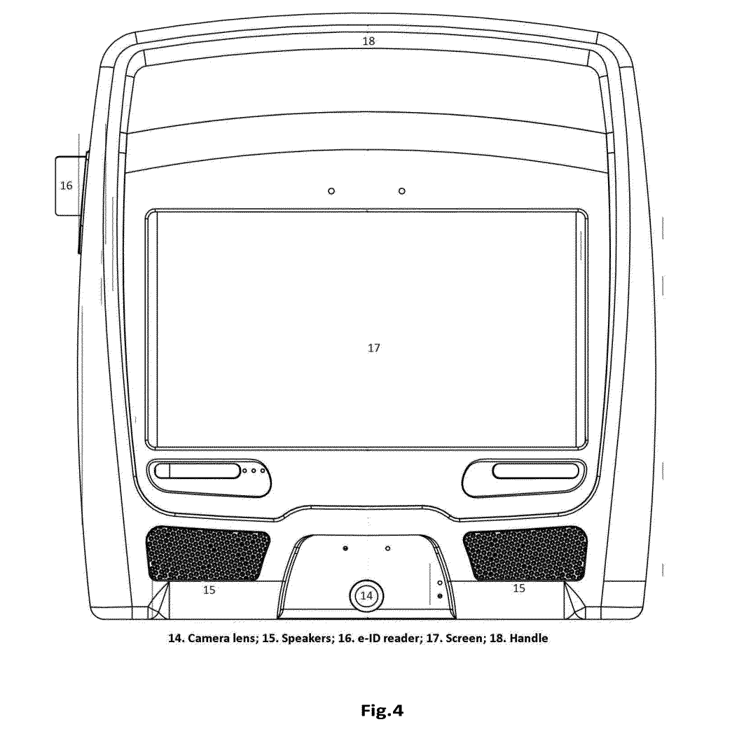

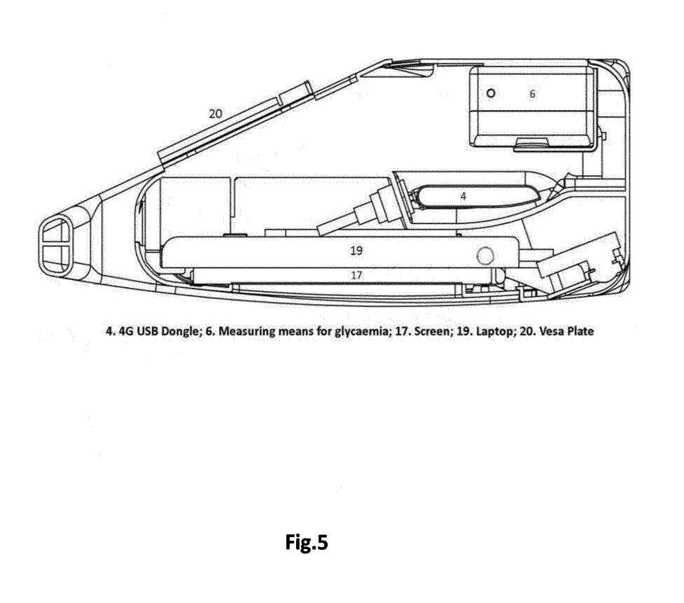

For illustration purposes, FIG. 4 and FIG. 5 show a front view and a side view respectively of a possible implementation of the portable system according to the present invention. In the front view, the camera lens (14), loudspeakers (15), screen (17), e-ID card reader (16) and handgrip (18) are visible. Handgrip is to be construed as a grip that allows carrying the system with one hand. In the side view, the vesa plate (20) to which the system is attached in this embodiment, the laptop (19), the modem (4)—in this example a 4G modem—and the measuring means (6) for determining the blood glucose level are shown along with the screen (17).

The portable system as proposed is a stand-alone system and can be used both inside the hospital and outside it, typically then in an ambulance or in the vicinity of a patient if the latter cannot be brought immediately into the ambulance.

The system can also be used for other pathologies in acute care. Examples of these include: cardiac diseases, traumatology or disaster medicine.

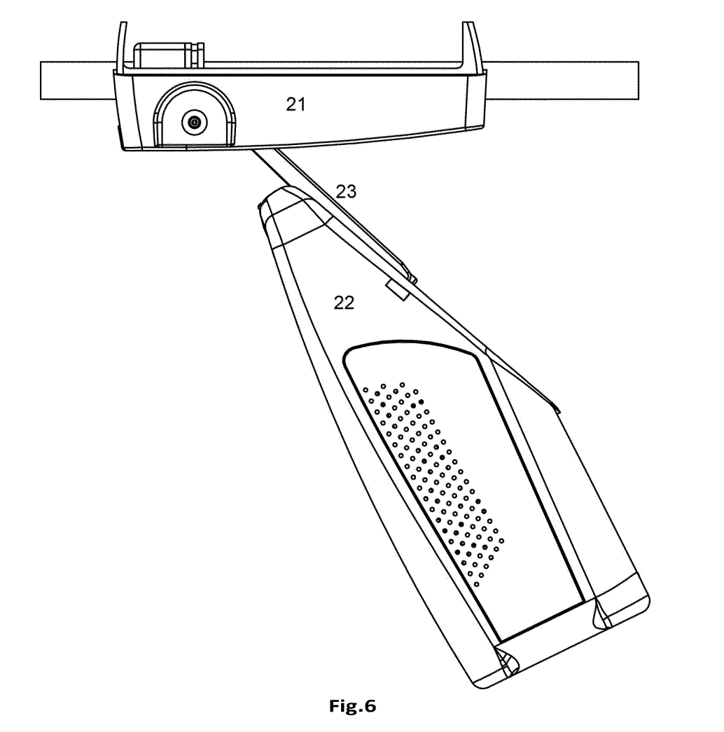

FIG. 6 illustrates an embodiment of the proposed system that also contains a safe and robust ambulance roof attachment. The unique properties and small space within an ambulance make it preferable that, when the housing of the system (22) is not attached to the roof of the ambulance, the arm (23) can safely and quickly be stored away into a roof housing (21). It is also preferable that the system is quickly attachable and detachable from the roof attachment and that the angle to the roof can be manipulated.

While the invention has been illustrated and described in detail in the drawings and foregoing description, such illustration and description are to be considered illustrative or exemplary and not restrictive. The foregoing description details certain embodiments of the invention. It will be appreciated, however, that no matter how detailed the foregoing appears in text, the invention may be practiced in many ways. The invention is not limited to the disclosed embodiments.

Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1-17. (canceled)

18. A portable system for remote diagnosis of a stroke, arranged in a housing provided with a handgrip and comprising
audio recording means for recording sound of a patient and audio reproduction means for reproducing sound for said patient,
portable video recording means for recording at least eye movements and foot movements of said patient, said portable video recording means arranged to be set up in such a way that the patient can be captured from head to toe,
measuring means to determine at least a blood glucose value in said patient,
communication means for forwarding said measured blood glucose value and said recorded movements and sound of said patient via a wireless network from the location of said patient and for receiving at least an audio signal originating from a location where a doctor is present.

19. The portable system according to claim 18, wherein said communication means are also configured to receive a video signal originating from the location where the doctor is present, and wherein the portable system comprises a monitor for visualising said image originating from the location where the doctor is present.

20. The portable system according to claim 18, wherein said portable video recording means can be set up with an angle of view of at least 80°.

21. The portable system according to claim 20, wherein said angle of view is at least 95°.

22. The portable system according to claim 19, wherein said monitor can be set up in such a way that the surface of said monitor forms an angle of at least 10° with the surface of said video recording means.

23. The portable system according to claim 18, wherein said measuring means are further equipped to determine at least the heart rate, blood pressure and/or arterial oxygen saturation, and wherein said communication means are equipped to forward the obtained measurement values.

24. The portable system according to claim 18, wherein said communication means are equipped to forward said image of said patient with a picture resolution of at least 160×120 pixels and a picture rate of at least 4 frames/second.

25. The portable system according to claim 18, comprising at least two independent batteries.

26. The portable system according to claim 25, wherein at least one of said batteries is provided with a voltage decharge controller.

27. The portable system according to claim 18, arranged for simultaneously activating at least said audio recording means, said portable video recording means and said communication means.

28. The portable system according to claim 18, comprising a reader for an electronic identity card.

29. The portable system according to claim 18, wherein said measuring means are further equipped to record an electrocardiogram and/or determine the blood clotting level, wherein said communication means are equipped to forward the obtained measurement values.

30. The portable system according to claim 18, comprising a processing unit configured to provide information on the health of said patient on the basis of measurements of at least two parameters.

31. The portable system according to claim 30, wherein said processing unit is equipped to perform a stroke scale.

32. The portable system according to claim 18, comprising a tripod to attach said video recording means.

33. The portable system according to claim 18, comprising a snap-on system and an arm to which said snap-on system can be attached.

34. A kit comprising a portable system according to claim 18 and one or more measuring means for determining vital parameters and/or a portable computer.
